(19) United States
(12) Patent Application Publication
(10) Pub. No.: US 2017/0128927 A1
(43) Pub. Date: May 11, 2017

(54) METHOD FOR SELF-REGULATION OF A SYSTEM

(71) Applicant: BASF SE, Ludwigshafen (DE)

(72) Inventors: Dominik GARELLA, Wiesloch (DE); Viviana SILVA, Limburgerhof (DE); Tobias LANG, Dossenheim (DE); Javier GARCIA PALACIOS, Koeln (DE)

(73) Assignee: BASF SE, Ludwigshafen (DE)

(21) Appl. No.: 15/321,993

(22) PCT Filed: Jun. 23, 2015
(86) PCT No.: PCT/EP2015/064094

§ 371 Date: Dec. 23, 2016

(30) Foreign Application Priority Data
Jun. 24, 2014 (EP) 14173612.4

(51) Int. Cl. B01J 35/00; B01J 8/00; B01J 8/02; C07C 209/36; B01J 8/18; B01J 8/08; B01J 23/00; B01J 23/755; B01J 8/42

(52) U.S. Cl. CPC B01J 35/0033; B01J 23/755; B01J 8/001; B01J 8/0285; B01J 8/42; B01J 8/1809; B01J 8/1836; B01J 8/087; B01J 8/082; B01J 23/002; C07C 209/36; B01J 2208/00017; B01J 2208/00663; B01J 2523/00

(57) ABSTRACT

The invention relates to a method for self-regulation of a system comprising the steps of:
(I) utilizing a magnetic field to transport magnetizable and/or magnetic particles out of a control volume or to localize said particles in the control volume,
(II) changing magnetic properties of the magnetizable and/or magnetic particles, which are ferromagnetic or paramagnetic, in the control volume by changing a temperature Tp of the magnetizable and/or magnetic particles or by changing the composition of the magnetizable and/or magnetic particles.

The invention relates to a method for self-regulation of a system, wherein a magnetic field is employed and the magnetic properties of magnetizable and/or magnetic particles are changed.

Magnetically enhanced methods utilize a magnetic field in order to improve individual steps of the method or indeed to implement them. Examples of magnetically enhanced processes are magnetically stimulated bioprocesses, alternating magnetic fields for heating, activation of free-radical reactions and also magnetic stirrers.

Magnetically enhanced reactors (MERs), in particular magnetically enhanced fluidized bed systems, are often described in the literature as attractive alternatives or additions to fixed bed reactors, fluidized bed reactors and suspension reactors also known as slurry reactors. Here, an external magnetic field acts on magnetizable and/or magnetic particles comprised in a fluidized bed. Magnetic properties of the magnetizable and/or magnetic particles depend, inter alia, on the chemical composition of the magnetizable and/or magnetic particles and/or on the temperature thereof. The fluidized bed systems can consist of gas-solid mixtures or gas-liquid mixtures. The simultaneous presence of gases, liquids and solids in one fluidized bed is likewise conceivable.

Magnetizable and/or magnetic particles having ferromagnetic properties are often employed in MERs as catalysts or as support materials for catalytically active substances fluidized in a continuously operated reactor. A fluid flowing through the reactor exerts a force on the magnetizable and/or magnetized particles in the MER in the flow direction of the fluid as is also the case for fluidized bed systems for example. On the other hand, magnetic forces of an applied magnetic field and normally also the gravitational force counteract this movement, induced by the flowing liquid, of the magnetizable and/or magnetic particles.

The advantages of magnetically enhanced fluidized beds are enhanced process control and, in comparison to fixed-bed reactors, a lower pressure drop and improved mass transfer and heat transfer and, in comparison to non-magnetically enhanced fluidized beds, reduced back-mixing, higher throughput, avoidance of bubble formation and bypasses of the fluid, the possibility of operation with smaller, up to nano-scale, particle sizes and reduced discharge and abrasion of the particles comprised in the fluidized bed. A magnetically enhanced fluidized bed can be used to combine advantages of a fixed bed, such as good mass transfer between fluid phase and solid phase and narrow residence time distribution of the phases, and advantages of a fluidized bed, such as high heat transfer coefficients and mass transfer coefficients, homogeneous temperature distribution and the possibility of continuous supply and removal of solid. The magnetic enhancement makes it possible to better control the movement of solid. Action of the magnetic field on the magnetic particles consequently provides an additional degree of freedom to influence the fluidization state of the magnetizable and/or magnetic particles. Using a magnetic field further provides alternatives to pneumatic conveying.

The interaction of changeable magnetic properties of the magnetizable and/or magnetic particles and a movement of the magnetizable and/or magnetic particles which, in turn, depends on the magnetic properties can be used for temperature management and reaction management in the reactor.

DE 10 2007 059 967 A1 discloses a method for carrying out chemical reactions with the aid of an inductively heated heating medium. To this end, the reaction medium is contacted with a solid heating medium heatable by electromagnetic induction. The heating medium can be ferromagnetic and can have a Curie temperature which deviates from the reaction temperature by no more than 20° C. The method thus provides self-regulation of the heating medium temperature and precludes overheating via the heating medium since the heating medium cannot be inductively heated to a temperature above the Curie temperature of the heating medium.

DE 696 03 270 T2 provides a magnetic separation method for iron carbide. The mixture of iron carbide, iron, iron oxides and admixed minerals withdrawn from the reaction space is separated by application of different temperatures and successively employed magnetic fields, utilizing the different Curie temperatures of the individual components of the mixture.

FR 2691718 A1 likewise describes separating iron-containing and non-iron-containing material with a magnetic field after cooling down to a temperature below the Curie temperature of the iron-containing material.

WO 2006/071527 discloses a reactor for synthesizing nanostructures, wherein ferromagnetic particles are employed which have a Curie temperature substantially equal to the reaction temperature and which comprise a catalyst. The particles are inductively heated.

DE 3642557 A1 describes a fluidized bed for pyrolyzing solid carbon-containing material, wherein magnetizable and/or magnetic particles are employed as heat transfer medium. The magnetizable and/or magnetic particles are heated separately and then supplied to the pyrolysis step. Following pyrolysis, the remaining solids are, if necessary, cooled down to a temperature below the Curie temperature of the magnetizable and/or magnetic particles. The magnetizable and/or magnetic particles are subsequently separated from the pyrolysis products using a magnetic field and the magnetizable and/or magnetic particles are recycled into the pyrolysis step.

U.S. Pat. No. 4,354,856 provides a method for recovering magnetic particles from a fluid. A ferromagnetic element is employed to attract the magnetic particles and is subsequently heated to a temperature greater than or equal to the Curie temperature of the ferromagnetic element in order to disengage the magnetic particles from the ferromagnetic element .

FR 2 676 374 A1 discloses a continuously operated magnetically enhanced particle bed, wherein the particles of the bed are replaced in an ongoing method by said particles being removed from the particle bed by suction and fresh particles being supplied to the particle bed.

EP 0 115 684 A1 discloses a method for carrying out reactions in a magnetically enhanced particle bed with a controlled reaction rate profile. The particles are magnetizable, can exhibit catalytic properties and are used for supply or removal of heat. To enhance conversion, reactivity and/or product yield, the particle bed is conveyed through the reactor in the form of a plug flow, optionally regenerated after emerging from the reactor and recycled into the reactor.

EP 0 021 854 A1, U.S. Pat. No. 4,294,688 and U.S. Pat. No. 4,292,171 each provide methods in which magnetizable, in some cases catalytically active, particles are continuously withdrawn from a magnetically enhanced fluidized bed and also regenerated and recycled.

DE 29 50 621 A1 describes a catalyst composition of passivated magnetic alloys for use in magnetically stabilized fluidized beds for high-temperature applications. The catalyst composition comprises aluminum, silicon and/or chromium, has a high Curie temperature and is stable in a corrosive environment.

The magnetically enhanced fluidized beds and magnetic field-based separation methods disclosed in the prior art documents do not take advantage of the dependence of the magnetic properties of a solid on the temperature or composition for self-regulation of a system, wherein transport of magnetizable and/or magnetic particles is used for temperature management or reaction management. For example in the method for temperature management described in EP 0 115 684 A1, a high level of process engineering complexity and outlay is required which externally controls the establishment of a desired temperature level by determining conveying rates. In U.S. Pat. No. 4,354,856, DE 696 03 270 T2 or DE 3642557 A1 for example, the change in the magnetic properties is brought about by changes outside the system in order that a separation principle is realizable at all. Although overheating by inductive supply of heat is prevented in DE 10 2007 059 967, it is, however, not possible to exert a self-regulating influence over the composition of the mixture in the reaction space here, in order to prevent overheating for reasons other than the inductive supply of heat.

It is an object of the present invention to provide a method for self-regulation of a system, wherein the composition of a mixture in a control volume is adjusted according to prevailing temperature conditions and/or prevailing reaction progress without being reliant upon an external closed loop control means, i.e., peripheral equipment outside the control volume.

The object is achieved by a method for self-regulation of a system comprising the steps of:

(I) utilizing a magnetic field to transport magnetizable and/or magnetic particles out of a control volume or to localize said particles in the control volume.

(II) changing magnetic properties of the magnetizable and/or magnetic particles, which are ferromagnetic or paramagnetic, in the control volume by changing a temperature Tp of the magnetizable and/or magnetic particles or by changing the composition of the magnetizable and/or magnetic particles.

The magnetic field is preferably an external magnetic field generated by various arrangements of coils, in particular electrical coils, known to those skilled in the art. In this connection, external means that the magnetic field is not generated in the control volume but the magnetic field exists in the control volume. Depending on the arrangement and configuration of the coils, the magnetic field exhibits different orientations, field strengths and homogeneities. The strength of a magnetic field H can be described by the following equation:

$$H = I \cdot \frac{n}{L} , \qquad ( 1 )$$

where I is current, n is the number of loops and L is the length of the coil. The unit of magnetic field strength H is Nm. Magnetic field strength is linearly correlated with magnetic induction B:

B=μ₀·μᵣ·H, (2)

where μ₀ is the magnetic field constant, i.e., the magnetic permeability of a vacuum, and μᵣ is relative permeability which is a measure of the influence on the magnetic field of the material comprised in the magnet.

The magnetic field exerts a magnetic force Fₘ on magnetic or magnetizable particles which depends on the particle properties and the properties of the external field:

Fₘ=μ₀·Vₚ·Mₚ·∇H (3)

where μ₀ is the magnetic field constant, Vₚ is the particle volume, Mₚ is the magnetization of the particle and ∇H is the gradient of the magnetic field strength at the location of the particle. The magnetization of the particle in turn depends on the magnetic field strength H of the external magnetic field:

Mₚ=κ·H (4).

Magnetic susceptibility κ is a measure of the magnetizability of material in an external magnetic field. In the case of ferri- and ferromagnetic materials, magnetic susceptibility κ depends, inter alia, on particle size, particle shape and field intensity. For diamagnetic and paramagnetic materials, magnetic susceptibility κ is constant.

It follows from combining equations (3) and (4) that the magnetic force Fₘ exerted by the magnetic field on magnetic or magnetizable particles having specific properties depends on the magnetic field strength H and the gradient ∇H of the magnetic field strength at the location of the particle in question:

Fₘ∝H·∇H (5).

The behavior of a magnetically enhanced fluidized bed is determined by the gravitational force F_{G}, the magnetic force Fₘ, flow resistance FR and uplift force F_{U}. The balance of forces for a single particle, neglecting collisions of the particles with one another, in a magnetically enhanced fluidized bed with an external magnetic field is given by:

F_{R}F_{U}=F_{G}Fₘ (6).

The actual influence of the magnetic field on the particles depends on the magnetic field strength H and the homogeneity of the magnetic field. Irrespective of the homogeneity of the field, the external magnetic field induces attractive forces between the individual particles which lead to aggregation and chain formation. The magnetic field generated in a volume occupied by the fluidized bed can be homogeneous or inhomogeneous. A homogeneous magnetic field exhibits field lines parallel to one another in the fluidized bed or in the reactor. In this case, the extent of fluidization in the fluidized bed decreases as a result of the magnetic field. The forces induced by the magnetic field bring about particle agglomeration and larger aggregates are therefore formed which behave like heavier particles in the fluidized bed. This is exploited in a magnetically enhanced fluidized bed. In an inhomogeneous magnetic field, which is generated using an array of different coils, the magnetic field lines are curved and a field line density relating to a cross-sectional area of the reactor is inconstant over a reactor height. A force similar to the gravitational force can be generated in the reactor when force vectors resulting from the magnetic field oppose an ascending fluid flow.

Different flow states can be generated in a reactor by varying the magnetic field strength. When the magnetic field strength is high the flow state approaches the flow state of a fixed bed and when the magnetic field strength is low the bed is completely fluidized. A magnetically enhanced fluidized bed can also be described as a partially fixed fluidized bed in a flowing fluid.

Magnetically enhanced reactors are generally operated with constant magnetic fields. Using alternating fields is likewise possible. Alternating fields can be used to generate a fluidized bed even when the flow rate of the fluid alone would not yet lead to fluidization.

In a further embodiment, screens and/or intermediate trays having hole diameters greater than the diameters of the magnetizable and/or magnetic particles can be employed in the control volume or at the edges of the control volume. Applying the magnetic field makes it possible for aggregates of the magnetizable and/or magnetic particles to form which, by contrast, have a diameter greater than the holes of the screens. This makes it possible to control the passage of the magnetizable and/or magnetic particles through the screens and/or intermediate trays using the magnetic field.

Axial fields and transverse fields are distinguished in terms of the orientation of the magnetic field lines in a fluidized bed, said field lines running parallel to the flow direction of the fluid in axial fields and perpendicularly to the flow direction of the fluid in transverse fields. It is preferable to use transverse magnetic fields, which can be generated via saddle coils or Helmholtz coils, since highly expanded fixed beds can be achieved and channel formation in the fluidized bed occurs less readily compared to when axial fields are used. Moreover, transverse fields have the advantage that a homogeneous magnetic field can exist in about 99% of the volume in which the magnetic field prevails. The volume fraction of a homogeneous magnetic field for axial fields is typically only about 30%. For axial fields, the magnetic field is homogeneous in its centre and the magnetic field strength increases in the direction of the windings of the coil. When a homogeneous magnetic field over the entire cross section of a reactor is desired, the coil diameter must be considerably larger than the reactor diameter in the case of axial fields. This gives rise to both increased material costs and a large operating volume and also increased energy requirements.

Magnetically stabilized fluidized beds often require a homogeneous magnetic field. In magnetically stabilized fluidized beds, interparticle magnetic forces cause aggregates to form which behave like relatively heavy particles in the fluidized bed and bring about more stable fluidization. By contrast, in a magnetically enhanced fluidized bed only weak forces occur between the individual particles.

The magnetically enhanced fluidized bed comprises magnetic or magnetizable particles and interaction between the magnetic field and the particles is therefore possible. The magnetically enhanced fluidized bed can comprise exclusively magnetic and/or magnetizable particles or a mixture of magnetizable and/or magnetic particles and non-magnetic or of non-magnetizable material. The mixture preferably comprises at least 10% by volume, in particular between 10% by volume and 30% by volume, of magnetic and/or magnetizable particles based on the bed volume. The magnetizable and/or magnetic particles preferably exhibit variable magnetic properties which can be paramagnetic or ferromagnetic. Paramagnetic materials have a permeability greater than 1. In paramagnetic materials, magnetic moments are arranged in a disordered fashion and partially order themselves in an external magnetic field for as long as the external magnetic field exists. Paramagnetic materials are drawn into an inhomogeneous magnetic field. They have a tendency to migrate into a stronger field. Compared to paramagnetic fields, magnetization of ferromagnetic materials can be described as stable. Ferromagnetic materials orient their elementary magnets parallel to one another. They can be attracted by a magnetic pole of an external magnetic field. Ferromagnetic materials exhibit an inherent magnetization in an external magnetic field. Examples of elements which are ferromagnetic at room temperature include iron, nickel and cobalt. The magnetic properties depend on the temperature Tp of the magnetizable and/or magnetic particles and/or on the composition of the magnetizable and/or magnetic particles. Depending on the temperature Tp of the magnetizable and/or magnetic particles, the magnetizable and/or magnetic particles are or are not under the influence of the magnetic field.

The Curie temperature Tc is the temperature of the magnetizable and/or magnetic particles above which ferromagnetic material becomes paramagnetic and thus loses its magnetic properties. In a magnetically enhanced fluidized bed, the magnetizable and/or magnetic particles can also be shielded from the influence of the magnetic field even when the temperature Tp of the magnetizable and/or magnetic particles has not yet reached the Curie temperature Tc when the magnetic force of the particles at this temperature Tp is small enough that the hydrodynamic forces in the system prevail and are the predominant forces for the movement of the magnetizable and/or magnetic particles.

The magnetizable and/or magnetic particles can be constructed in different ways. They can, for example, consist of composite materials or have a core-shell construction, wherein a magnetizable core has a non-magnetic material coating as described, for example, in Dong et al., Selective acetylene hydrogenation over core-shell magnetic Pd-supported catalysts in a magnetically stabilized bed, American Institute of Chemical Engineers Journal, 2008, volume 54, number 5, pages 1358 to 1364, and Fu et al., Preparation and properties of magnetic alumina microspheres with a γ-Fe₂O₃/SiO₂ core and Al₂O₃ shell, Journal of Natural Gas Chemistry, 2011, volume 20, number 1, pages 72 to 76. The non-magnetic material can comprise a catalytically active substance or a catalytically active substance can be immobilized on the non-magnetic coating.

Cu—Ni ferrites are preferred materials for the magnetizable and/or magnetic particles since they allow desired Curie temperatures Tc to be adjusted through the choice of the copper to nickel ratio and, moreover, they are catalytically active materials for hydrogenation reactions such as the hydrogenation of nitrobenzene, described in WO 2008/034770 A1 for example.

Typical Curie temperatures Tc of magnetizable and/or magnetic particles employed are between 300° C. and 900° C. The Curie temperature Tc of mixed materials depends on the methods by which the mixed materials are produced. A preferred mixed material for magnetizable and/or magnetic particles comprises iron, copper and nickel. The molar ratio of nickel to copper is preferably between 4 and 0.25. A preferred method of producing the magnetizable and/or magnetic particles comprises mixing starting materials and calcining at a temperature of at least 900° C. over a period of several hours. It is preferable when the magnetizable and/or magnetic particles do not contain a crystalline magnetite phase. This is advantageous since the Curie temperature Tc of magnetite is comparatively high with values between 560° C. and 590° C. and thus is higher than, for example, a target reaction temperature range for the hydrogenation of nitrobenzene of 250° C. to 350° C.

TABLE 1
Examples for materials having different Curie temperatures

| Material | Curie temperature [° C.] |
|---|---|
| Co | 1121 |
| Fe | 770 |
| Ni | 358 |
| Magnetite (Fe₃O₄) | 578 |
| γ-Fe₃O₄ (maghemite) | 858 |
| Fe—Ni (46% by weight of Ni) | 733 |
| Fe—Co (50% by weight of Co) | 998 |
| Li ferrite | 631 |
| Ni ferrite | 575 to 597 |
| Co ferrite | 495 to 520 |
| Cu ferrite | 410 to 490 |
| Mn ferrite | 295 to 303 |
| Ni_{0.8}Cu_{0.2}Fe₂O₄ | 436 |
| Ni_{0.6}Cu_{0.4}Fe₂O₄ | 401 |
| Ni_{0.4}Cu_{0.6}Fe₂O₄ | 336 |
| Ni_{0.2}Cu_{0.8}Fe₂O₄ | 285 |

For the purposes of the invention, the Curie temperature Tc of the magnetizable and/or magnetic particles is the Curie temperature Tc of at least one component of the magnetizable and/or magnetic particles. The magnetizable and/or magnetic particles can simultaneously comprise different components having different Curie temperatures Tc. In such a case, what is meant is the Curie temperature Tc which in the course of the method exceeds or is exceeded by the temperature Tp of the magnetizable and/or magnetic particles or which can exceed or be exceeded thereby. The magnetizable and/or magnetic particles are preferably phase-pure and a resulting Curie temperature Tc is therefore homogeneously distributed over the magnetizable and/or magnetic particles. Homogenation can, for example, be achieved during particle production by a final high-temperature treatment at about 1000° C. with a subsequent cold shock.

Movement of the magnetizable and/or magnetic particles into the control volume or out of the control volume can be effected by various forces. In preferred embodiments, the magnetizable and/or magnetic particles are transported by the flowing fluid, the gravitational force or the magnetic field.

In one embodiment, the magnetizable and/or magnetic particles are transported by the flowing fluid which can be gaseous or liquid. The movement of the magnetizable and/or magnetic particles is in the flow direction of the fluid here and is caused by friction between the fluid and the surface of the magnetizable and/or magnetic particles. Here, the magnetizable and/or magnetic particles can enter the control volume with the fluid and/or through a separate feed point and are then localized in the control volume on account of the magnetic field when the magnetizable and/or magnetic particles have ferromagnetic properties. Likewise, magnetizable and/or magnetic particles can be discharged from the control volume, in which the magnetic field prevails, with a flowing fluid when they have paramagnetic properties.

In a further embodiment, a magnetic field prevails only outside or at the edge of the control volume. The magnetic field outside the control volume can be used to remove magnetizable and/or magnetic particles having ferromagnetic properties from the control volume.

In an alternative embodiment, magnetizable and/or magnetic particles having ferromagnetic properties can also be removed from the control volume by application of a moving magnetic field in the control volume to convey the magnetizable and/or magnetic particles. To this end, a plurality of coils are arranged on top of one another or side by side. The magnetizable and/or magnetic particles are aggregated by the magnetic field and transported by switching on and switching off of adjacent coils since the adjacent magnetic fields overlap and do not terminate immediately when the associated coil is switched off.

In further embodiments and depending on vertical spatial arrangement, magnetizable and/or magnetic particles can be transported into the control volume or out of the control volume when said particles are not yet or no longer under the influence of the magnetic field which can be in the control volume or outside the control volume. For instance, gravitational force can be used to transport magnetizable ferromagnetic particles into the control volume where they are localized by a magnetic field or gravitational force can be used to transport magnetizable paramagnetic particles out of a control volume where they had been localized by a magnetic field until the magnetic properties changed from ferromagnetic to paramagnetic, for example on account of temperature changes or a change in composition. In this connection, localized means that the magnetic field prevents the ferromagnetic magnetizable and/or magnetic particles from exiting the control volume. This does not preclude movement of the ferromagnetic magnetizable and/or magnetic particles in the control volume.

In a preferred embodiment, ferromagnetic magnetizable and/or magnetic particles have been localized in the control volume by a magnetic field. The temperature Tp increases over the course of the method on account of processes taking place in the control volume, for example exothermic chemical reactions, adsorption or addition of substances having a temperature higher than the initial temperature Tp, until the temperature Tp is greater than or equal to the Curie temperature Tc of the magnetizable and/or magnetic particles. When the Curie temperature Tc is reached, the ferromagnetic magnetizable and/or magnetic particles become paramagnetic, are no longer under the influence of the magnetic field and are transported out of the control volume. Here, the transport of the magnetizable and/or magnetic particles out of the control volume can be used to remove heat from the system. Thus, mass transfer can be coupled to heat transfer. Moreover, a magnetocaloric effect occurs when the properties of the magnetizable and/or magnetic particles shift from ferromagnetic to paramagnetic. The magnetic moments in the magnetizable and/or magnetic particles are no longer oriented by the magnetic field. A second order phase transition takes place. The specific heat capacity, i.e., the second derivative of specific enthalpy with respect to temperature, of the magnetizable and/or magnetic particles changes abruptly and said particles suddenly cool down. This is advantageous in this embodiment in relation to temperature management with the aim of heat removal.

In an alternative embodiment, paramagnetic magnetizable and/or magnetic particles are in the control volume. The temperature Tp, which is initially higher than the Curie temperature Tc of the magnetizable and/or magnetic particles, decreases over the course of the method until the temperature Tp is lower than the Curie temperature Tc. This occurs, for example, on account of an endothermic chemical reaction taking place in the control volume or due to heat losses via the edges of the control volume by convection or heat conduction or due to the addition of substances having a temperature lower than the initial temperature Tp. When the Curie temperature Tc is reached, the paramagnetic magnetizable and/or magnetic particles become ferromagnetic and are now under the influence of a magnetic field which transports the now ferromagnetic magnetizable and/or magnetic particles out of the control volume. The control volume can be chosen such that the now ferromagnetic magnetizable and/or magnetic particles are transported into a zone in the fluidized bed used for heat input. The magnetic field can be either a static magnetic field prevailing outside the control volume or a moving magnetic field prevailing in the control volume. Here, in turn, transport into the control volume of magnetizable and/or magnetic particles having a temperature higher than the temperature Tp of the magnetizable and/or magnetic particles in the control volume can be used to supply heat to the system. To compensate, and to reduce the mass to be heated in the control volume, cooled-down magnetizable and/or magnetic particles can then be removed from the control volume as herein described.

In a preferred embodiment, a chemical reaction is carried out in the control volume. A chemical reaction is a reaction in which a transformation of matter takes place. The chemical reaction can be an exothermic or an endothermic reaction.

When the chemical reaction is an exothermic reaction, reaction energy is released which can contribute to increasing the temperature Tp. In a preferred embodiment, the temperature increase in the control volume brought about by the exothermic reaction is limited by magnetizable and/or magnetic particles, which have paramagnetic properties since their Curie temperature has been exceeded, being transported out of the control volume as described hereinabove.

In a preferred embodiment, the chemical reaction is a strongly exothermic reaction. Reactions are described as strongly exothermic when they have a reaction enthalpy value greater than 200 kJ/mol, preferably greater than 300 kJ/mol and, in particular, greater than 350kJ/mol.

The method according to the invention can, in particular, be used advantageously for temperature management of heterogeneously catalyzed exothermic reactions since this makes it possible to ensure sufficient heat removal tailored to the course of the reaction. Insufficient heat removal can lead to the formation of hot spots, i.e., local temperature increases, undesired temperature profiles and to thermal runaway, i.e., overheating of the reactor. These phenomena exacerbate, for example, byproduct formation, catalyst deactivation or coking. Furthermore, ensuring operational safety is a basic requirement for carrying out exothermic reactions which depends on controlled temperature management.

When the chemical reaction is an endothermic reaction, heat is removed from the system due to the reaction progressing and this can result in a reduction in the temperature Tp.

In a preferred embodiment, the Curie temperature Tc of the magnetizable and/or magnetic particles has been matched to the specific chemical reaction carried out in the control volume, said reaction being defined by, inter alia, at least one participating reactant, at least one participating product and a reaction temperature Tr. The reaction temperature Tr is the temperature at which the chemical reaction is carried out. The reaction temperature Tr is preferably chosen after optimization in view of process engineering considerations. The Curie temperature Tc of the magnetizable and/or magnetic particles is preferably between 100° C. and 900° C., more preferably between 350° C. and 400° C. It is particularly preferable when the Curie temperature Tc of the magnetizable and/or magnetic particles deviates from the reaction temperature Tr of the chemical reaction by no more than 150 K, in particular no more than 50 K and optimally no more than 20 K. When the magnetizable and/or magnetic particles comprise different components with different Curie temperatures, the Curie temperature Tc of the magnetizable and/or magnetic particles is to be understood as meaning the Curie temperature Tc closest to the reaction temperature Tr.

In a further embodiment, the magnetizable and/or magnetic particles catalyze the chemical reaction or comprise a material which is catalytically active with respect to the chemical reaction. In this case when the magnetizable and/or magnetic particles are transported out of the control volume, the chemical reaction carried out there is slowed down or stopped. When the chemical reaction is simultaneously an exothermic reaction, this limits the evolution of heat in the control volume.

The magnetic properties of the magnetizable and/or magnetic particles can depend not only on temperature but also or alternatively on the composition of the magnetizable and/or magnetic particles. Thus, for example, different oxidation states of at least one component of the magnetizable and/or magnetic particles can have different magnetic properties. The chemical reaction can change the oxidation states of at least one component of the magnetizable and/or magnetic particles.

When the magnetizable and/or magnetic particles catalyze the chemical reaction or comprise a material catalytically active with respect to the chemical reaction and catalyst activation during the chemical reaction and/or catalyst activation during a regeneration step changes the composition of the magnetizable and/or magnetic particles such that the magnetic properties of the particles also change, the extent of catalyst deactivation and/or catalyst activation determines the location, which can be in the control volume or outside the control volume, of the magnetizable and/or magnetic particles. In this embodiment, the system can self-regulate by deactivated catalyst being transported out of the control volume and/or activated catalyst being transported into the control volume. For example, a catalyst in the synthesis of styrene has different magnetic properties in deactivated form to the same catalyst in non-deactivated form, for example iron in different oxidation states. In a further example, organic sulfur compounds and hydrogen sulfide can be cleaved in a first method step as follows:

FeS+R—SH→FeS₂+R—H

FeS+H₂S→FeS₂+H₂.

The first method step can be carried out in a fluidized bed or in another solid conveying reactor for example. A liquid phase and/or a gas phase comprising a desulfurized product can subsequently be removed from the solid FeS₂ and the FeS₂ can be conveyed into a regenerator. Retrocleavage to FeS can be effected in the regenerator at high temperatures. Elemental sulfur S can be removed and FeS can be recycled into the first method step.

In an alternative embodiment, the magnetizable and/or magnetic particles are a reactant or product of the chemical reaction and the magnetic properties of the reactant are distinct from the magnetic properties of the corresponding product and, in each case, one is ferromagnetic and the other is paramagnetic. In this case too, the change in the magnetic properties of the magnetizable and/or magnetic particles can be linked to the change in oxidation state due to the chemical reaction of at least one component of the magnetizable and/or magnetic particles. This makes it possible, for example, to localize a ferromagnetic reactant in the control volume using the magnetic field until it is converted into the paramagnetic product and then transported out of the control volume as product on account of the abovementioned forces. This optimizes the residence time of the reactant in the control volume such that the reactant remains in the control volume until it has been converted to the product.

Alternatively, a paramagnetic reactant can be converted into a ferromagnetic product in the control volume, said product then being transported out of the control volume by a magnetic field outside the control volume or by a moving magnetic field in the control volume. One advantage of this embodiment is that the reaction is directly combined with a separation method and an often required subsequent separation of product and unconverted reactant is rendered unnecessary. It is, moreover, possible to increase the conversion as a ferromagnetic reactant remains in the control volume until it has been converted and is only then under the influence of the magnetic field and is not discharged from the control volume prematurely while still in the form of a reactant. It is assumed here that reaction-promoting conditions prevail in the control volume, for example in terms of temperature and the presence of a catalyst.

In a further embodiment, once transported out of the control volume the magnetizable and/or magnetic particles are cooled or heated and/or regenerated and then optionally recycled into the control volume. The magnetizable and/or magnetic particles can consequently serve as heat-transfer medium or can enhance process stability or catalyst productivity as deactivated catalyst can be selectively removed from the control volume and activated catalyst can be supplied to the control volume.

When an exothermic reaction is carried out, the system preferably consists of a magnetically enhanced reactor (MER) and a cooler. The Curie temperature Tc of the magnetizable and/or magnetic particles, which preferably comprise a catalyst of the exothermic reaction, is preferably adjusted according to the desired reaction temperature Tr using appropriate production methods. Magnetizable and/or magnetic particles which are heated up in the MER, where they are localized by the magnetic field, to a temperature Tp higher than their Curie temperature Tc lose their magnetic properties and are conveyed to the cooler. After cooling down to a temperature Tp lower than their Curie temperature Tc the magnetizable and/or magnetic particles behave magnetically again and are returned to the reactor. This temperature control of exothermic reactions makes it possible to avoid hot spots and runaway reactions, heat transfer and selectivity are improved and catalyst deactivation is reduced.

For cooling or heating, the magnetizable and/or magnetic particles can be supplied to a region under appropriate temperature control or they can be contacted with a heat exchanger device. Depending on the reactor concept, the particles can be supplied to the temperature control step using the flow of the fluid or the magnetic force.

In a further embodiment, the method is used to safeguard a reactor in which an exothermic reaction is carried out. Here, the exothermic reaction is carried out at a control reaction temperature Tcr which is lower than a threshold temperature Tt, the Curie temperature Tc of the magnetizable and/or magnetic particles has been adjusted such that the Curie temperature Tc of the magnetizable and/or magnetic particles is higher than the reaction temperature Tr and lower than the threshold temperature Tt and a difference between the Curie temperature Tc of the magnetizable and/or magnetic particles and the threshold temperature Tt is no more than 20° C. and the magnetic properties of the magnetizable and/or magnetic particles only change when the reaction temperature Tr, which is initially equal to the control reaction temperature Tcr, increases causing the temperature Tp of the magnetizble and/or magnetic particles to increase and the temperature Tp of the magnetizable and/or magnetic particles to exceed the Curie temperature Tc. The magnetizable and/or magnetic particles preferably catalyze the exothermic reaction or comprise a catalytically active material. When the exothermic reaction is carried out as planned at the control reaction temperature Tcr, the magnetic properties of the magnetizable and/or magnetic particles remain unchanged. The control reaction temperature Tcr is the temperature at which the reaction is carried out in accordance with the design of the method. The Curie temperature Tc of the magnetic particles has been matched to the reaction conditions and the employed apparatuses, in particular to the control reaction temperature Tcr and the threshold temperature Tt, such that in the event of any incidents leading to an unplanned temperature increase in the control volume the catalyst is discharged from the control volume on account of the change in the magnetic properties of the magnetizable and/or magnetic particles. This makes it possible to prevent overheating of the reactor also known as thermal runaway of the reactor. This method for safeguarding an exothermic reaction ensures that evolution of heat is reduced or stopped and simultaneously heat coupled to mass transfer is removed from the control volume by the catalyst being separated from the reactants as soon as the prevailing reaction temperature Tr and thus also the temperature Tp of the magnetizable and/or magnetic particles exceeds the Curie temperature Tc of the magnetizable and/or magnetic particles. It is preferable when the Curie temperature Tc of the magnetizable and/or magnetic particles has been adjusted such that it has been geared to the threshold temperature Tt of the reaction system. The threshold temperature Tt is the temperature above which damage due to overheating would be expected and the magnetizable and/or magnetic particles are to be discharged from the control volume and/or reactor. The threshold temperature Tt is system-specific and, inter alia, depends on the reactor material which determines the pressure and temperature resistance of the reactor and on any side reactions which could take place at elevated temperatures and which are to be avoided. The difference between the Curie temperature Tc of the magnetizable and/or magnetic particles and the threshold temperature Tt is no more than 100° C., preferably no more than 50° C., more preferably no more than 20° C. and most preferably no more than 10° C. The control reaction temperature Tcr is preferably between 100° C. and 900° C., more preferably between 200° C. and 600° C. and most preferably between 250° C. and 350° C. In this embodiment, the method according to the invention does not proceed under planned method conditions but the necessary precautions have been taken to allow the method according to the invention to proceed when there is an unplanned temperature increase in the reactor.

The control volume can be part of any reactor type known to the skilled person. In a preferred embodiment, the control volume is comprised in a fixed-bed reactor, in an expanded-bed reactor which is also known as a moving-bed reactor and which, in comparison to a fixed bed reactor, facilitates movement of the solids in the bed, in a suspension reactor, in a fluidized-bed reactor or in a reactor which comprises a magnetically enhanced fluidized bed. Particularly preferred are a fluidized-bed reactor and a reactor comprising a magnetically enhanced fluidized bed.

It is conceivable to apply the method according to the invention to many different types of reaction systems such as caprolactam purification, production of ethylene from acetylene, high-temperature Fischer-Tropsch synthesis, ammonium synthesis, flue gas desulfurization, biodiesel production, dehalogenation in wastewater treatment, synthesis gas production comprising the separation of hydrogen and carbon dioxide, oligomerization of olefins, production of hydrogen from methane, hydrocarbon reforming, separation of olefins and paraffins, catalytic reduction of nitricoxide, carbon monoxide methanation and diphenyl carbonate synthesis. Other fields of application are further petrochemical and environmental technology processes and also bioprocess engineering processes comprising enzymes immobilized on magnetizable and/or magnetic particles and other reactions and separation processes such as chromatography and drying.

In a preferred embodiment, the chemical reaction is a hydrogenation, preferably production of an aromatic amine and more preferably conversion of nitrobenzene to aniline. For aniline production, the method according to the invention provides an alternative or an addition, for example, to a heat transfer medium which is integrated into the fluidized bed and which serves to remove heat from the exothermic reaction.

### BRIEF DESCRIPTION OF THE FIGURES

Operative examples of the invention are shown in the figures and will be more particularly described in the description which follows.

FIG. 1 is a schematic diagram of an exothermic reaction in a magnetically enhanced fluidized bed with self-regulating temperature management.

FIG. 2 is a schematic diagram of an exothermic reaction in a magnetically enhanced fluidized bed with self-regulating temperature management and recycling of magnetizable and/or magnetic particles.

FIG. 3 is a schematic diagram of an exothermic reaction in a batch reactor with alternating magnetic field and self-regulating temperature management.

FIG. 4 is a schematic diagram of an endothermic reaction in a fluidized bed with self-regulating temperature management.

FIG. 5 is a schematic diagram of an endothermic reaction in a fluidized bed with self-regulating temperature management and lateral heating element.

FIG. 6 is a schematic diagram of an endothermic reaction in a batch reactor with self-regulating temperature management.

FIG. 7 is a schematic diagram of an endothermic reaction in a batch reactor with self-regulating temperature management and magnetic conveying.

FIG. 8 shows conversions of nitrobenzene in the presence of magnetizable and/or magnetic particles.

FIG. 9 shows temperature profiles for heating of magnetizable and/or magnetic particles.

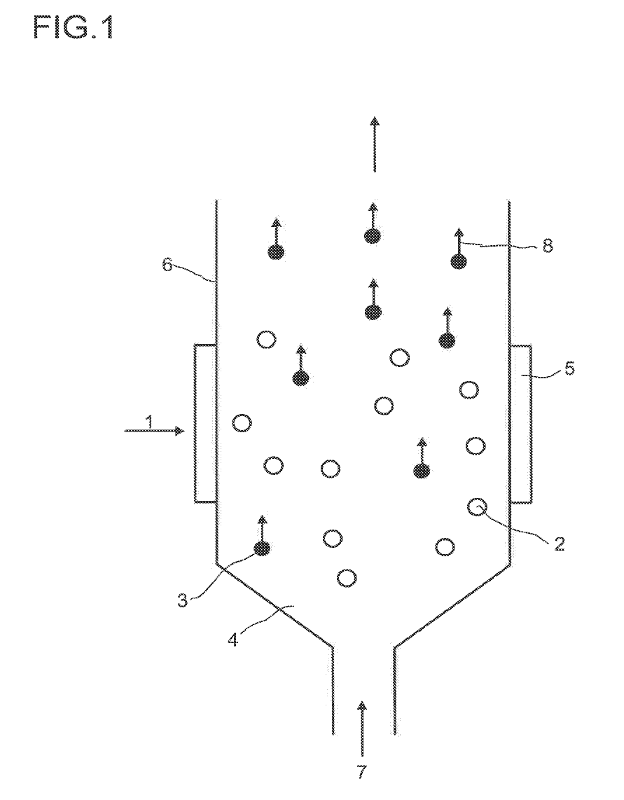

FIG. 1 is a schematic diagram of an exothermic reaction in a magnetically enhanced fluidized bed with self-regulating temperature management.

FIG. 1 shows a first fluidized bed 1 in which an exothermic reaction is carried out. To this end, a fluid phase 4 comprising at least one reactant flows through the first fluidized bed 1 which is bounded by a reactor wall 6. The first fluidized bed 1 comprises fluidized magnetizable and/or magnetic particles. The magnetizable and/or magnetic particles are catalytically active with respect to the exothermic reaction carried out in the first fluidized bed 1. At least one magnet coil 5 is used to generate a static magnetic field in the region of the first fluidized bed 1 and said field stabilizes the fluidized bed and exerts on magnetic particles a force acting in a direction opposite to the flow direction 7 of the fluid phase 4. A first portion 2 of the magnetizable and/or magnetic particles has a temperature Tp2 lower than the Curie temperature Tc of the particles. Thus, the first portion 2 of the magnetizable and/or magnetic particles is under the influence of the applied magnetic field. The movement of the first portion 2 of the magnetizable and/or magnetic particles in the first fluidized bed 1 is directionless and the first portion 2 of the magnetizable and/or magnetic particles remains in the first fluidized bed 1, has been localized in the first fluidized bed 1. A second portion 3 of the magnetizable and/or magnetic particles has a temperature Tp3 higher than the Curie temperature Tc of the particles. Thus, the second portion 3 of the magnetizable and/or magnetic particles is not influenced by the magnetic field. The force which is generated by the magnetic field and which acts in a direction opposite to the flow direction 7 is not applicable to the second portion 3 of the magnetizable and/or magnetic particles which is therefore discharged from the first fluidized bed 1 in flow direction 8. The temperature management in the first fluidized bed 1 is self-regulating. The progress of the heterogeneously catalyzed exothermic reaction raises the temperature in the fluidized bed and consequently also the temperature Tp of the magnetizable and/or magnetic particles and individual particles from the first portion 2 of the magnetizable and/or magnetic particles therefore pass into the second portion 3 of the magnetizable and/or magnetic particles, lose the influence of the magnetic field, are carried off by the fluid phase 4 and discharged from the first fluidized bed 1. This reduces the amount of catalyst in the first fluidized bed 1 and thus also reduces the reaction rate and the rate of temperature increase which, moreover, can also be influenced and controlled externally via the inflow of the fluid phase 4. Particles of the second portion 3 of the magnetizable and/or magnetic particles, which have been discharged from the first fluidized bed 1, may be cooled down and recycled into the fluidized bed.

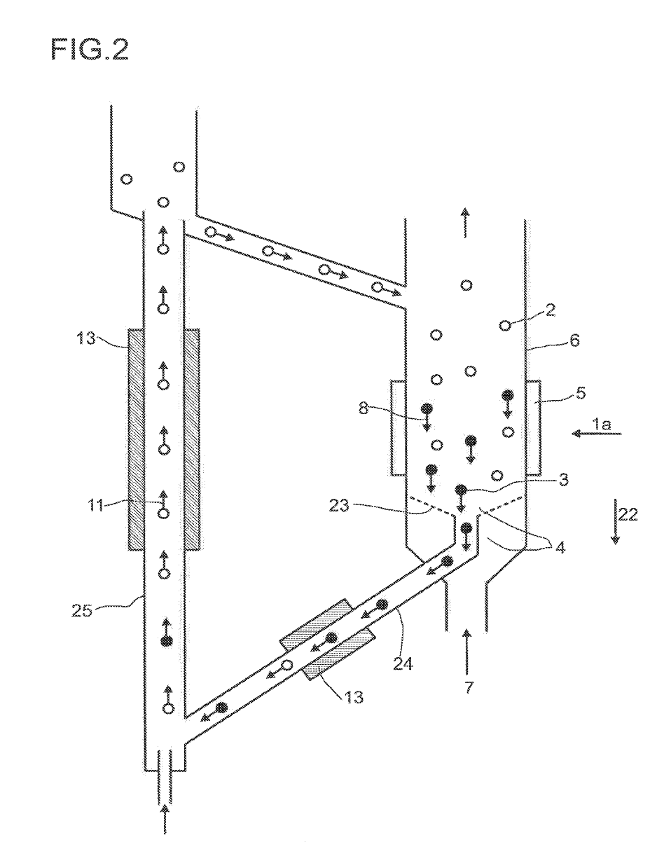

FIG. 2 is a schematic diagram of an exothermic reaction in a magnetically enhanced fluidized bed with self-regulating temperature management and recycling of magnetizable and/or magnetic particles.

A second fluidized bed la shown in FIG. 2 differs from the first fluidized bed 1 shown in FIG. 1 in terms of the flow rate of the fluid phase 4 through the fluidized bed. The flow rate of the fluid phase 4 in FIG. 2 is lower and the second portion 3 of the magnetizable and/or magnetic particles, which is not under the influence of the magnetic field, therefore follows the gravitational force and sinks. In addition, the at least one magnet coil 5 is used to generate a magnetic field which exerts on magnetic particles a force acting in the flow direction 7 of the fluid phase 4. When the Curie temperature Tc of individual magnetizable and/or magnetic particles is exceeded, these exit the second fluidized bed la via a downpipe 24. The occasional magnetizable and/or magnetic particle of the first portion 2 could also enter the downpipe. However, the proportion of particles of the second portion 3 in the downpipe is generally greater than the proportion of particles of the first portion 2. A grid 23 surrounds the circumference of the downpipe 24 at the point of entry of the downpipe 24 into the second fluidized bed la and said grid connects the circumference of the downpipe 24 with the reactor wall 6. The fluid phase 4 can flow through the grid 23, while the magnetizable and/or magnetic particles cannot pass through the grid and the magnetizable and/or magnetic particles moving downward in their flow direction 8 are directed toward the downpipe 24. In the downpipe 24, the magnetizable and/or magnetic particles of the second portion 3 of the magnetizable and/or magnetic particles are directed past at least one cooling jacket 13 and, as a result, said particles cool down, their temperature falls below their Curie temperature Tc and they pass into the first portion 2 of the magnetizable and/or magnetic particles. The cooled-down magnetizable and/or magnetic particles are returned via a riser pipe 25 to the second fluidized bed la where they are again under the influence of the magnetic field until they are reheated as described in connection with FIG. 1 to a temperature above their Curie temperature Tc by the heterogenerously catalyzed exothermic reaction carried out in the second fluidized bed 1a.

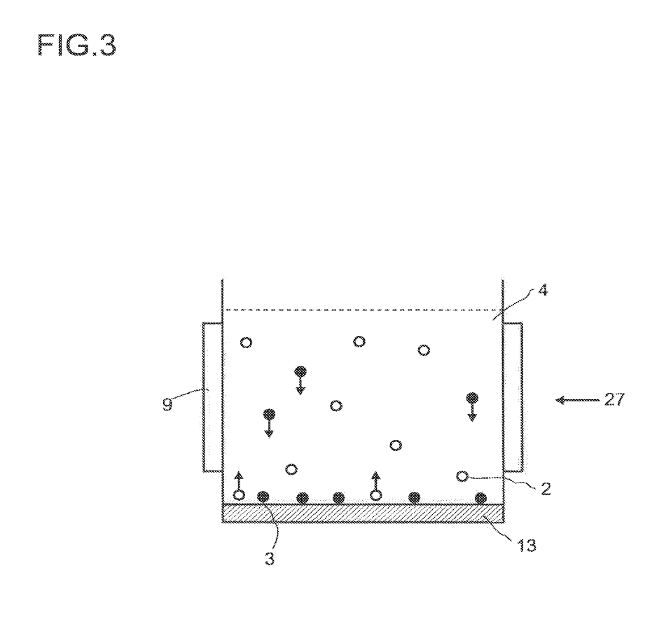

FIG. 3 is a schematic diagram of an exothermic reaction in a batch reactor with alternating magnetic field and self-regulating temperature management.

In accordance with FIG. 3, in a first batch reactor 27 an exothermic reaction is carried out in a fluid phase 4 which comprises dispersed magnetizable and/or magnetic particles. At least one magnet coil 9 generates an alternating magnetic field which moves magnetic particles and ensures commixing in the first batch reactor 27. A first portion 2 of the magnetizable and/or magnetic particles, which has a temperature Tp2 lower than the Curie temperature Tc, is under the influence of the alternating magnetic field and is fluidized in the fluid phase. The progress of the exothermic reaction causes the temperature in the first batch reactor 27 and the temperature of the magnetizable and/or magnetic particles to increase and, as a result, individual magnetizable and/or magnetic particles of the first portion 2 are heated to a temperature Tp3 higher than their Curie temperature Tc and consequently pass into the second portion 3 of the magnetizable and/or magnetic particles. The second portion 3 of the magnetizable and/or magnetic particles is not under the influence of the alternating magnetic field and sinks downward. At the bottom of the first batch reactor 27 there is a cooling jacket 13 and sedimented particles of the second portion 3 of the magnetizable and/or magnetic particles come into contact with said cooling jacket and are cooled to a temperature Tp2 lower than their Curie temperature Tc. Now, these cooled-down particles are again under the influence of the alternating magnetic field and are again fluidized in the fluid phase 4. Magnetizable and/or magnetic particles of the second portion 3 are thus supplied to the cooling jacket 13 and cooled down in a self-regulating fashion. Magnetizable and/or magnetic particles of the first portion 2 are removed from the cooling jacket 13 in a self-regulating fashion as soon as their temperature falls below the Curie temperature Tc.

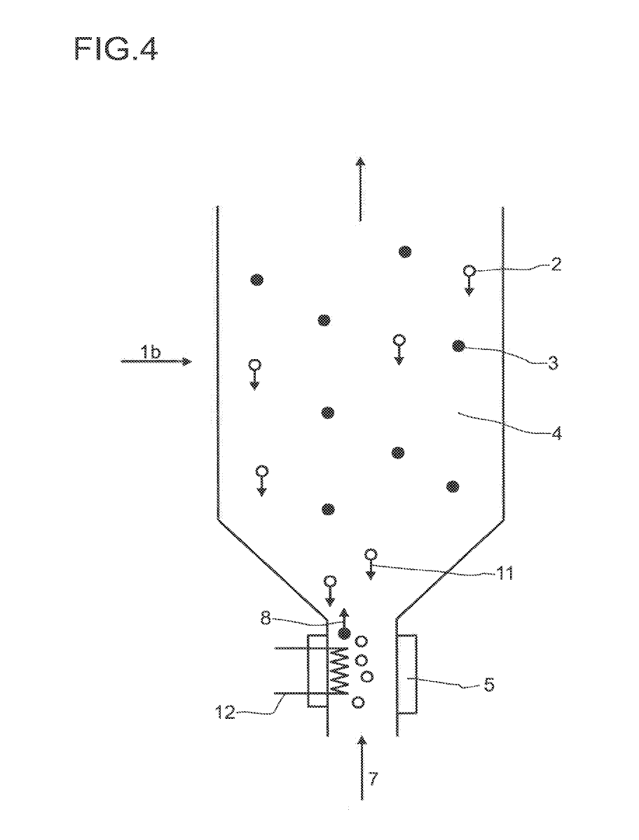

FIG. 4 is a schematic diagram of an endothermic reaction in a fluidized bed with self-regulating temperature management.

In FIG. 4, an endothermic reaction is carried out in a third fluidized bed 1b. A heating element 12 and at least one magnet coil 5 are disposed at the feed point of the fluid phase 4 below the third fluidized bed 1b. The third fluidized bed 1b initially comprises a second portion 3 of magnetizable and/or magnetic particles at a temperature Tp3 higher than the Curie temperature Tc of the particles. A feed point of the fluid phase 4 into the third fluidized bed lb is preferably chosen such that the second portion 3 of the magnetizable and/or magnetic particles does not reach the heating element 12 and the at least one magnet coil 5 due to a fluid velocity in flow direction 7 in a cross section at the height of heating element 12 and the at least one magnet coil 5 exceeding the discharge velocity of the second portion 3 of the magnetizable and/or magnetic particles at a point of entry into the third fluidized bed 1b. The progress of the endothermic reaction reduces the temperature of the magnetizable and/or magnetic particles and the temperature of individual magnetizable and/or magnetic particles falls below the Curie temperature Tc of said particles and they therefore pass into the first portion 2 of the magnetizable and/or magnetic particles and are under the influence of the magnetic field generated by the at least one magnet coil 5. The first portion 2 of the magnetizable and/or magnetic particles is attracted by the at least one magnet coil 5 and, on account of the now operative force additionally exerted by the magnetic field on the first portion 2 of the magnetizable and/or magnetic particles, moves in the direction of the at least one magnet coil 5 and the heating element 12 which are arranged such that the magnetic field of the at least one magnet coil 5 positions the magnetizable and/or magnetic particles of the first portion 2 in contact with or in proximity to the heating element 12. The magnetizable and/or magnetic particles positioned there are heated to a temperature Tp3 above their Curie temperature Tc by the heating element 12, thus pass into the second portion 3 of the magnetizable and/or magnetic particles, are no longer under the influence of the magnetic field and are transported in flow direction 7 back into the third fluidized bed 1b by the flowing fluid phase 4. When there is a secondary feed point of the fluid phase 4, at half of the height of the third fluidized bed 1b for example, a plurality of zones can be formed in the fluidized bed. Here, the method according to the invention can be employed to achieve a desired particle circulation in the different zones.

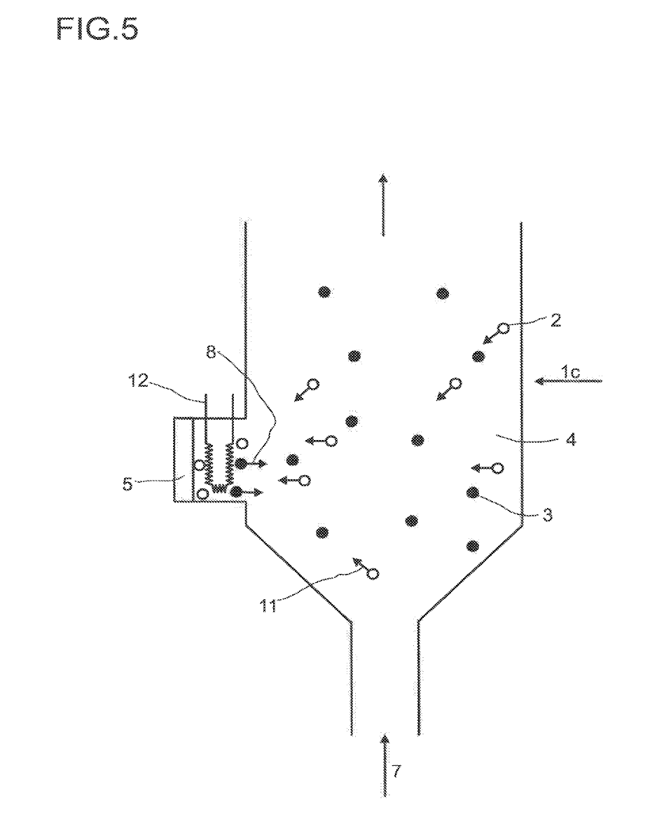

FIG. 5 is a schematic diagram of an endothermic reaction in a fluidized bed with self-regulating temperature management and lateral heating element.

A fourth fluidized bed 1c shown in FIG. 5 differs from the third fluidized bed 1b in that the heating element 12 and the at least one magnet coil 5 are disposed to the side of the fourth fluidized bed 1c. The directions of movement 8 and 11 of the magnetizable and/or magnetic particles 3 and of the magnetizable and/or magnetic particles 2 are thus not parallel to the flow direction 7 of the fluid phase 4. Moreover, in one embodiment of the fourth fluidized bed 1c the magnetizable and/or magnetic particles 3 can also coincidentally move in the direction of the at least one magnet coil 5 which is preferably prevented in the fluidized bed 1b described hereinabove due to the fluid velocity in the region of the at least one magnet coil 5 being high enough that particles can only arrive at the at least one magnet coil 5 and at the heating element 12 under the influence of the magnetic field.

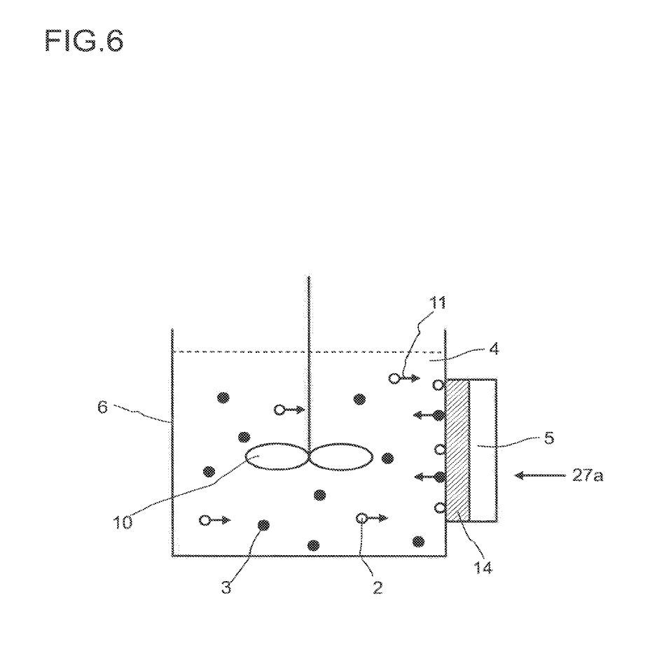

FIG. 6 is a schematic diagram of an endothermic reaction in a batch reactor with self-regulating temperature management.

In accordance with FIG. 6, in a second batch reactor 27a an endothermic reaction is carried out in a fluid phase 4 comprising dispersed magnetizable and/or magnetic particles. Commixing is achieved via stirrer 10. At least one magnet coil 5 and a heating jacket 14 are together disposed on the reactor wall 6 such that a first portion 2 of the magnetizable and/or magnetic particles, which particles have a temperature Tp2 lower than their Curie temperature Tc, is under the influence of the magnetic field generated by the at least one magnet coil 5 and is drawn to the heating jacket 14. The progress of the endothermic reaction reduces the temperature Tp3 of the magnetizable and/or magnetic particles and the temperature of individual magnetizable and/or magnetic particles of the second portion 3 falls below the Curie temperature Tc of said individual particles and, as a result, they pass into the first portion 2 of the magnetizable and/or magnetic particles and are under the influence of the magnetic field generated by the at least one magnet coil 5. The first portion 2 of the magnetizable and/or magnetic particles is attracted by the at least one magnet coil 5 and moves in the direction of the at least one magnet coil 5 and the heating jacket 14 which are arranged such that the magnetic field of the at least one magnet coil 5 positions the magnetizable and/or magnetic particles of the first portion 2 in contact with or in proximity to the heating jacket 14. The magnetizable and/or magnetic particles positioned there are heated to a temperature Tp3 above their Curie temperature Tc by the heating jacket 14, thus pass into the second portion 3 of the magnetizable and/or magnetic particles, are no longer under the influence of the magnetic field and are carried off again by the stirred fluid phase 4 and removed from the heating jacket 14 and dispersed in a self-regulating fashion according to their temperature.

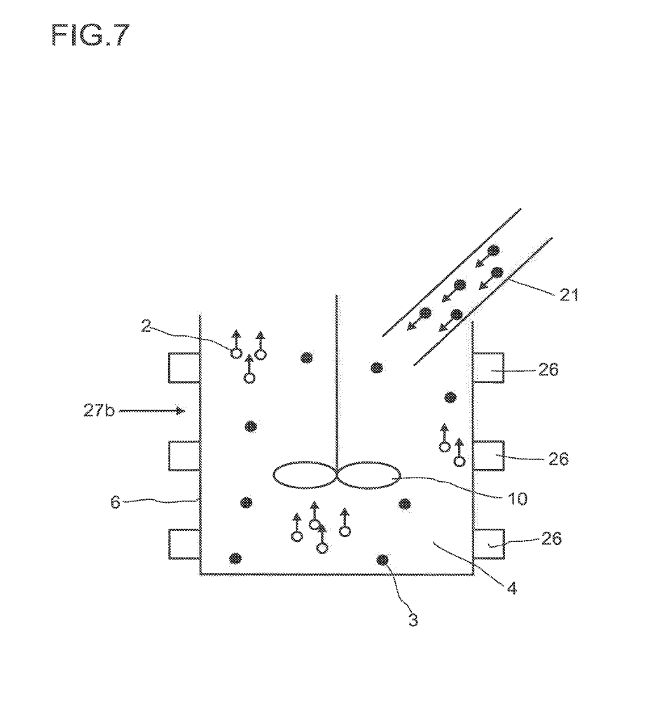

FIG. 7 is a schematic diagram of an endothermic reaction in a batch reactor with self-regulating temperature management and magnetic conveying.

An endothermic reaction is carried out in a third batch reactor 27b, shown in FIG. 7, in the manner of FIG. 6. However, magnet coils 26 for generating magnetic fields of a magnetic conveying system are disposed on the reactor wall 6 in place of the at least one magnet coil 5 and the heating jacket 14. A first portion 2 of the magnetizable and/or magnetic particles is under the influence of the magnetic fields of the magnet coils 26 and is removed from a reaction mixture consisting at least of the fluid phase 4 and the magnetizable and/or magnetic particles by means of the magnetic conveying system. Outside the third batch reactor 27b, the magnetizable and/or magnetic particles of the first portion 2 can be reheated to a temperature Tp3 above their Curie temperature Tc and recycled into the third batch reactor 27b via a feed line 21 for example. Alternatively or in addition, fresh magnetizable and/or magnetic particles at a temperature Tp3 above their Curie temperature Tc can be supplied to the third batch reactor 27b via the feed line 12.

In addition to the embodiments of FIGS. 1 to 7, further embodiments arise due to the magnetizable and/or magnetic particles, i.e., both the first portion 2 and the second portion 3, exhibiting no catalytic activity but rather being employed merely for heat transfer or due to the magnetic properties of the magnetizable and/or magnetic particles being brought about via a change in their composition such as change in the oxidation state in place of a temperature change.

### Example 1 Production of a Solid for Use in Magnetic Particles

Three samples, M1, M2 and M3, were produced in different ways. All samples comprised iron oxide, copper oxide and nickel oxide. All samples consisted of 67.3% by weight of Fe₂O₃, 20.11% by weight of CuO and 12.59% by weight of NiO. To produce M1, the components Fe₂O₃, CuO and NiO were mixed in the stated ratio, milled in a mortar and calcined at 900° C. for 9 hours. To produce M2, NiCO₃.2Ni(OH)₂, CuCO₃.Cu(OH)₂ and Fe(NO₃)₃.9H₂O were milled, dried, initially at 120° C. for 16 hours then at 150° C. for 16 hours, and subsequently thermally decomposed, at 250° C. for 2 hours, then at 400° C. for 2 hours and then at 550° C. for 2 hours, to retain the corresponding oxides. The sample was then calcined at 900° C. for 9 hours. To produce M3, the corresponding metal hydroxides were co-precipitated with KOH at a pH of 10 and a temperature of 80° C. The sample was then dried at 100° C. for 16 hours and calcined at 350° C. for 4 hours. In order to enhance the hardness of the samples, all materials were tabletted and subsequently milled to a particle size of between 100 μm and 300 μm.

### Example 2 Catalytic Activity of the Magnetic Particles

The catalytic activity of samples M2 and M3 was analyzed. To this end, in each case 2.5 g of a sample were charged to a glass bottle and 90 g of nitrobenzene and 90 g of aniline were added thereto. The mixture was purged with nitrogen in an autoclave and subsequently heated to 130° C. under 35 bar of hydrogen pressure and with stirring at 200 rpm. As soon as the temperature of 130° C. was reached, the stirring speed was increased to 1300 rpm in order to start the reaction.

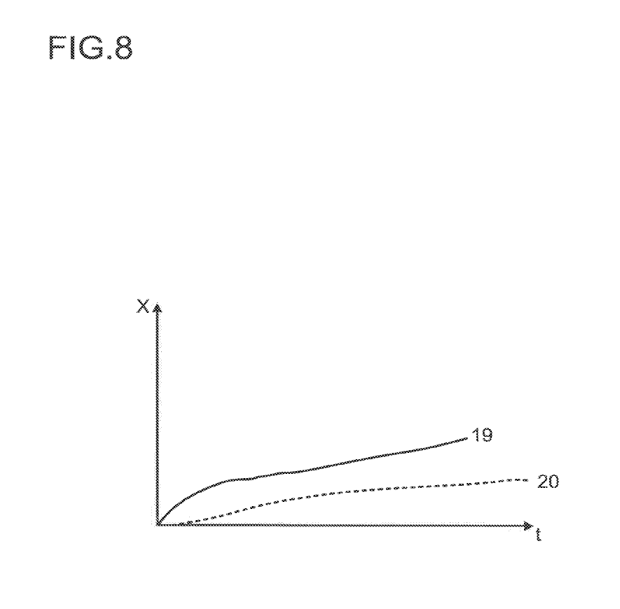

The results are shown in FIG. 8. To this end, the conversion 17 of nitrobenzene in percent was plotted as a function of time t in hours. Both the conversion shown in graph 19 for sample M3, and the conversion shown in graph 20 for sample M2 increased over time, a higher conversion being achieved in the same time for sample M3. The Ni—Cu ferrites analyzed consequently showed activity with respect to the hydrogenation of nitrobenzene.

### Example 3 Changing the Magnetic Properties of Magnetic Particles

In order to observe the magnetic properties of the samples qualitatively, a pendulum experiment was carried out. A pendulum of non-magnetic material was filled with magnetic sample material. The pendulum was displaced from its rest position using a magnetic field generated by a permanent magnet. The pendulum displaced from its rest position was heated with hot air at a temperature of 600° C. generated by a gas burner. The temperature of the sample was continuously measured with a thermocouple which simultaneously served as a pendulum mounting. The temperature of the permanent magnet was assumed to be constant.

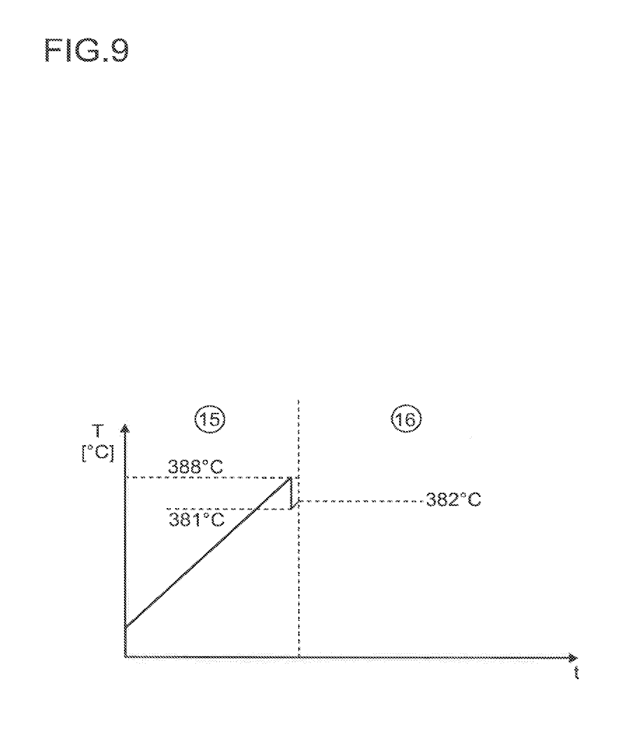

As soon as the Curie temperature of the sample material had been exceeded due to the heating with hot air, the sample material lost its magnetic properties and the pendulum returned to its rest position since it was no longer influenced by the permanent magnet. The temperature profile over time tin minutes for sample M1 is shown in FIG. 9. The temperature of sample M1 initially increased continuously up to 388° C. at which point it fell abruptly to 381° C. and then rose to 382° C. again. The influence of the permanent magnet on the sample and the contact between the pendulum and the permanent magnet were lost at this point. The sample is under the influence of the permanent magnet in region 15 shown in the figure; this is not the case in region 16 shown in the figure. The abrupt temperature change is attributable to the magnetocaloric effect which occurs as soon as the Curie temperature is reached. The Curie temperature of sample M1 was thus determined as approximately 388° C.

Disengagement of the pendulum from the permanent magnet in this manner is successful when the Curie temperature of the sample material is approximately between 350° C. and 400° C. Sample M2 could not be disengaged from the permanent magnet by supplying heat as described and the Curie temperature of sample M2 was determined as 540° C. by alternative methods of measurement.

Sample M3 showed no magnetic properties toward the permanent magnet and was not attracted to the permanent magnet.

The Curie temperature of sample M2 was quantitatively determined by thermogravimetric analysis using a permanent magnet, dynamic differential calorimetry and a high-frequency inductance measurement.

Depending on the manner of production, the three samples M1, M2 and M3 exhibited different magnetic properties. It was possible to influence the Curie temperature of the solids produced. Furthermore, M1 and M2 were calcined again at a higher temperature which made it possible to influence the Curie temperatures. The reduction in the Curie temperature achieved by calcining resulted from restructuring processes in the sample.

### Comparative Example 1 Production of Aniline

The production of aniline from nitrobenzene is carried out using a copper catalyst as described in WO 2010/130604 A2 for example. The exothermic reaction is carried out in a fluidized-bed reactor with internal heat exchanger. A fluid comprising hydrogen as reactant flows through the fluidized bed. The nitrobenzene is injected into the fluidized bed in liquid form. Heat transfer is the limiting factor when the reaction is carried out in this way.

### Example 4 Production of Aniline

The reaction is carried out as in comparative example 1, but a magnetically enhanced fluidized bed is employed in place of a fluidized-bed reactor with internal heat exchanger and the catalyst is comprised in magnetic catalyst particles. The Curie temperature Tc of the magnetic catalyst particles is 350° C. When the magnetic particles have a temperature lower than their Curie temperature, the magnetic particles are localized in the fluidized bed with the aid of an applied magnetic field. When the magnetic particles in the fluidized bed are heated to a temperature higher than their Curie temperature, the magnetic particles are discharged from the fluidized bed with the fluid phase. This discharges heat and catalyst mass from the fluidized bed with the magnetic particles. Here, the amount of heat discharged is self-regulating since more heat is discharged the more magnetic particles are heated to a temperature higher than their Curie temperature Tc.

### LIST OF REFERENCE NUMERALS

1 first fluidized bed

1a second fluidized bed

1b third fluidized bed

1c fourth fluidized bed

2 first portion of magnetizable and/or magnetic particles at a temperature Tp2 lower than the Curie temperature Tc of the particles

3 second portion of magnetizable and/or magnetic particles at a temperature Tp3 higher than the Curie temperature Tc of the particles

4 fluid phase

5 magnet coil

6 reactor wall

7 flow direction of the fluid phase 4

8 direction of movement of the second portion 3 of the magnetizable and/or magnetic particles

9 magnet coil for generating an alternating magnetic field

10 stirrer

11 direction of movement of the first portion 2 of the magnetizable and/or magnetic particles

12 heating element

13 cooling jacket

14 heating jacket

15 region in which the sample is under the influence of the permanent magnet

16 region in which the sample is not under the influence of the permanent magnet

19 graph for sample M3

20 graph for sample M2

21 feed line

22 gravitational force

23 grid

24 downpipe

25 riser pipe

26 magnet coil for generating magnetic fields of a magnetic conveying system

27 first batch reactor

27a second batch reactor

27b third batch reactor

X conversion of nitrobenzene in [%]

t time in [h]

1-18. (canceled)

19. A method for self-regulation of a system, the method comprising:
(I) utilizing a magnetic field to transport magnetizable and/or magnetic particles out of a control volume or to localize said particles in the control volume, wherein the magnetizable and/or magnetic particles:
a. are localized in the control volume by the magnetic field, when said particles have ferromagnetic properties, and transported out of the control volume by a flowing fluid or gravity, when said particles have paramagnetic properties, or
b. are transported out of the control volume by the magnetic field, when said particles have ferromagnetic properties, and are localized in the control volume, when said particles have paramagnetic properties, and
(II) changing magnetic properties of the magnetizable and/or magnetic particles, which are ferromagnetic or paramagnetic, in the control volume by changing a temperature Tp of the magnetizable and/or magnetic particles or by changing the composition of the magnetizable and/or magnetic particles,
wherein at least one chemical reaction is carried out in the control volume.

20. The method according to claim 19, wherein the magnetic field is a moving magnetic field.

21. The method according to claim 19, wherein the temperature Tp increases and the magnetizable and/or magnetic particles are transported out of the control volume when their temperature Tp is higher than their Curie temperature Tc.

22. The method according to claim 19, wherein the at least one chemical reaction is an exothermic reaction.

23. The method according to claim 22, wherein the temperature Tp increases and at least part of the energy for elevating the temperature Tp is liberated by the exothermic reaction.

24. The method according to claim 19, wherein the temperature Tp decreases and the magnetizable and/or magnetic particles are transported out of the control volume when their temperature Tp is lower than their Curie temperature Tc.

25. The method according to claim 19, wherein the at least one chemical reaction is an endothermic reaction.

26. The method according to claim 25, wherein the temperature Tp decreases and at least part of the energy emitted by the magnetizable and/or magnetic particles is utilized for carrying out the endothermic reaction.

27. The method according to claim 19, wherein the magnetizable and/or magnetic particles catalyze the at least one chemical reaction or comprise a catalytically active material.

28. The method according to claim 19, wherein the magnetizable and/or magnetic particles are a reactant or a product of the at least one chemical reaction or comprise a reactant or a product of the at least one chemical reaction.

29. The method according to claim 19, wherein once transported out of the control volume the magnetizable and/or magnetic particles are cooled or heated and/or regenerated and recycled into the control volume.

30. The method according to claim 19, wherein the composition of the magnetizable and/or magnetic particles changes by the at least one chemical reaction changing an oxidation state of at least one component of the magnetizable and/or magnetic particles.

31. The method according to claim 19, wherein the control volume is part of a fixed bed reactor, an expanded bed reactor, a fluidized bed reactor or a suspension reactor.

32. The method according to claim 19, wherein the at least one chemical reaction is a hydrogenation.

33. The method according to claim 19, wherein a difference between the Curie temperature Tc of the magnetizable and/or magnetic particles and a reaction temperature Tr of the at least one chemical reaction is not more than 150 K.

34. The method according to claim 22, wherein the exothermic reaction is carried out at a control reaction temperature Tcr which is lower than a threshold temperature Tt, the Curie temperature Tc of the magnetizable and/or magnetic particles has been adjusted such that the Curie temperature Tc of the magnetizable and/or magnetic particles is higher than the reaction temperature Tr and lower than the threshold temperature Tt and a difference between the Curie temperature Tc of the magnetizable and/or magnetic particles and the threshold temperature Tt is not more than 20° C. and the magnetic properties of the magnetizable and/or magnetic particles only change when the reaction temperature Tr, which is initially equal to the control reaction temperature Tcr, increases causing the temperature Tp of the magnetizble and/or magnetic particles to increase and the temperature Tp of the magnetizable and/or magnetic particles to exceed the Curie temperature Tc.

35. The method according to claim 23, wherein the exothermic reaction is carried out at a control reaction temperature Tcr which is lower than a threshold temperature Tt, the Curie temperature Tc of the magnetizable and/or magnetic particles has been adjusted such that the Curie temperature Tc of the magnetizable and/or magnetic particles is higher than the reaction temperature Tr and lower than the threshold temperature Tt and a difference between the Curie temperature Tc of the magnetizable and/or magnetic particles and the threshold temperature Tt is not more than 20° C. and the magnetic properties of the magnetizable and/or magnetic particles only change when the reaction temperature Tr, which is initially equal to the control reaction temperature Tcr, increases causing the temperature Tp of the magnetizble and/or magnetic particles to increase and the temperature Tp of the magnetizable and/or magnetic particles to exceed the Curie temperature Tc.

36. The method according to claim 30, wherein the exothermic reaction is carried out at a control reaction temperature Tcr which is lower than a threshold temperature Tt, the Curie temperature Tc of the magnetizable and/or magnetic particles has been adjusted such that the Curie temperature Tc of the magnetizable and/or magnetic particles is higher than the reaction temperature Tr and lower than the threshold temperature Tt and a difference between the Curie temperature Te of the magnetizable and/or magnetic particles and the threshold temperature Tt is not more than 20° C. and the magnetic properties of the magnetizable and/or magnetic particles only change when the reaction temperature Tr, which is initially equal to the control reaction temperature Tcr, increases causing the temperature Tp of the magnetizble and/or magnetic particles to increase and the temperature Tp of the magnetizable and/or magnetic particles to exceed the Curie temperature Tc.
